(19) United States
(12) Patent Application Publication
(10) Pub. No.: US 2021/0220131 A1
(43) Pub. Date: Jul. 22, 2021

(54) TRANSSEPTAL DELIVERY SYSTEM AND METHODS FOR THERAPEUTIC DEVICES OF THE AORTIC VALVE

(71) Applicant: Synecor LLC, Durham, NC (US)

(72) Inventors: Richard S Stack, Chapel Hill, NC (US); William L. Athas, Chapel Hill, NC (US); Kevin W. Johnson, Durham, NC (US)

(21) Appl. No.: 17/214,899

(22) Filed: Mar. 28, 2021

Related U.S. Application Data
(63) Continuation-in-part of application No. 16/860,015, filed on Apr. 27, 2020.
(60) Provisional application No. 62/811,369, filed on Feb. 27, 2019.
(60) Provisional application No. 63/043,072, filed on Jun. 23, 2020.

(51) Int. Cl. A61F 2/24; A61B 17/34

(52) U.S. Cl. CPC A61F 2/2427; A61B 2017/003; A61B 17/3468

(57) ABSTRACT

A method for delivering a therapeutic device to a target aortic valve site includes transseptally positioning a cable to run between afemoral vein, through the heart via the right atrium, left atrium, mitral valve, aortic valve and into the aorta and arterial vasculature with the flexible cable having a first end extending outside the body from a venous vessel and a second end external to the patient at the femoral artery. The aortic valve therapeutic device is advanced over the cable from outside the body and introduced into a femoral artery. A steerable sheath is advanced over the cable, into the venous vasculature and into the left ventricle of the heart. The aortic valve therapeutic device is pushed in a distal direction from the femoral artery to advance the aortic valve therapeutic device to the target site. The sheath protects surrounding tissues from the cable during movement of the therapeutic device through the vasculature, and is used to steer the therapeutic device from the front during final positioning to coaxially align the aortic valve therapeutic device at the target site while avoiding contact of the therapeutic device with the subvalvular conduction system.

This application is a continuation in part of U.S. application Ser. No. 16/860,015, filed Apr. 27, 2020 which claims the benefit of U.S. Provisional Application No. 62/811,369, filed Feb. 27, 2019. This application also claims the benefit of U.S. Provisional Application No. 63/043,072, filed Jun. 23, 2020. Each of the above applications is incorporated herein by reference.

### BACKGROUND

Transcatheter aortic valve replacement (TAVR) delivery systems are used to deliver replacement aortic valves to the aortic valve annulus using an intravascular approach. There are certain challenges associated with use of currently available TAVR delivery systems. In some patients, imprecise non-orthogonal placement of the TAVR device in the aortic valve annulus can cause paravalvular leak (PVL) and complete heart block (CHB). Impingement on the septum during valve expansion can create injury to the His bundle, resulting in the need for a permanent pacemaker. Precise positioning and orientation of the TAVR valve at the target site is highly desirable for avoiding such potential complications.

Commonly owned co-pending U.S. application Ser. No. 16/365,601 (Ref: AEG-1120) describes a transseptal delivery system for driving aortic valve therapeutic devices (AVTD's) such as TAVR delivery systems into place using a combination of pulling force, pushing force, steering force and momentum. A related system that is used instead for transeptally driving mitral valve therapeutic devices into place is described in Applicant's co-pending application Ser. No. 16/396,677 (Ref: ATR-830). Another co-pending U.S. application Ser. No. 16/578,373 (Ref: SYNC-5000) describes a transseptal delivery system and method that may be used to deliver percutaneous ventricular assist devices, or other devices such as aortic valve therapeutic devices or mitral valve therapeutic devices to their target locations.

Each of the above-described applications is incorporated herein by reference.

The present application describes a method of using a system that is similar to that described in U.S. application Ser. No. 16/578,373 for delivering an aortic valve therapeutic device, such as a TAVR delivery system carrying a TAVR valve, to an aortic valve site using a modified approach to the aortic valve site. In the present application, the therapeutic device is introduced into the vasculature on the arterial side (e.g. via the right femoral artery “RFA”) vs the venous side as described in each of the co-pending applications. The system and method described in this application allows the TAVR delivery system to be precisely maneuvered coaxially into the center of the native or a prosthetic aortic valve, orthogonal to the aortic valve annulus and away from the subvalvular conduction system

### BRIEF DESCRIPTION OF THE DRAWINGS

FIGS. 1-3F show components of a first embodiment of a conveyor system, in which:

FIG. 1 is a side elevation view of a Right-to-Left conduit (“RLC”);

FIG. 2A is a side elevation view of a conveyor cable;

FIG. 2B is a cross-section view of the conveyor cable taken along the plane marked 2B-2B in FIG. 2A;

FIG. 3A is a perspective view of a left ventricle redirector (“LVR”);

FIG. 3B is a side elevation view of the portion of the left ventricle redirector encircled in FIG. 3A.

FIGS. 3C and 3D are side elevation views of alternative embodiments of distal tip sections for the LVR, each shown in the curved position.

FIG. 3E is a perspective view of the distal tip section of FIG. 3B in the straight position.

FIG. 3F is a cross-section view of the LVR of FIG. 3A, taken along the plane designated 3F-3F in FIG. 3E.

FIG. 3G is an exploded view of the handle of the LVR shown in FIG. 3A.

FIGS. 3H-3J are a sequence of drawings schematically illustrating movement of the pull wire slider during activation and locking of the pull wires.

FIG. 3K is similar to FIG. 3J but shows only the pull wires and the engagement feature of the slider.

FIG. 3L is similar to FIG. 3K but shows an alternative configuration of pull wires and engagement features for the slider.

FIGS. 4-18 are a sequence of drawings schematically illustrating use of the system of FIGS. 1-3E in which:

FIG. 4 illustrates transseptal catheterization.

FIG. 5 illustrates advancement of the RLC over the wire;

FIG. 6 illustrates the RLC after it has been advanced over the wire into the left ventricle and as it is advancing over the wire into the ascending aorta;

FIG. 7 shows advancement of the wire and RLC into the descending aorta;

FIG. 8 shows the cable having replaced the wire through the RLC, and use of a snare to withdraw the cable from the right femoral artery;

FIG. 9 shows the cable after the RLC has been removed, before the LVR is advanced over the cable at the right femoral vein;

FIG. 10 shows the LVR after it has been advanced over the cable and into the left ventricle and after the RLC has been withdrawn;

FIG. 11 is similar to FIG. 10 but shows the LVR in its deployed position in the left ventricle and the TAVR delivery system after it has been advanced over the cable at the right femoral artery and advanced into the aortic arch;

FIGS. 12 and 13 are similar to FIG. 11, but show the TAVR delivery system traversing the aortic arch;

FIGS. 14-17 are similar to FIG. 13, but show the TAVR delivery system being advanced into the aortic valve annulus as it is oriented and steered from its distal tip, using the steering features of the LVR to apply traction to the distal end of the TAVR delivery system;

FIG. 18 shows the TAVR valve at the implant site following removal of the LVR, cable and TAVR delivery system.

### DETAILED DESCRIPTION

The presently disclosed system is designed to aid in the delivery of a TAVR device, other aortic valve therapeutic device or cardiac therapeutic devices to a location within the heart, such as at the aortic valve.

As will be appreciated from a review of the more detailed discussion that follows, rather than simply pushing a non-steerable TAVR delivery system over a guidewire or relying on a bend of the TAVR delivery device from behind the housing with pull wires, the presently disclosed system directly steers the TAVR delivery system and guides it forward from the front of the device. This unique approach gives greater control over the movement of the TAVR delivery system through the aorta and into the heart and allows precising steering of the TAVR device into position for deployment in the aortic valve annulus. As it enters the aortic valve annulus, the TAVR delivery system (and thus the TAVR device it carries) may be positioned precisely through the use of a unique bridging guy wire steering mechanism present in a device referred to as the left ventricle redirector or “LVR.” (described in detail below).

In the description of the system and method below, the access points for the components of the system are described as the right femoral vein (“RFV”) for the venous access and the right femoral artery (“RFA”) for the arterial access. However, the system and method can just as readily be used with a different combination of venous and arterial access, including the left femoral vein and artery (“LFV”, “LFA”), right subclavian vein and artery (“RSV”, “RSA”), left subclavian vein and artery, or the right or left internal jugular vein.

System

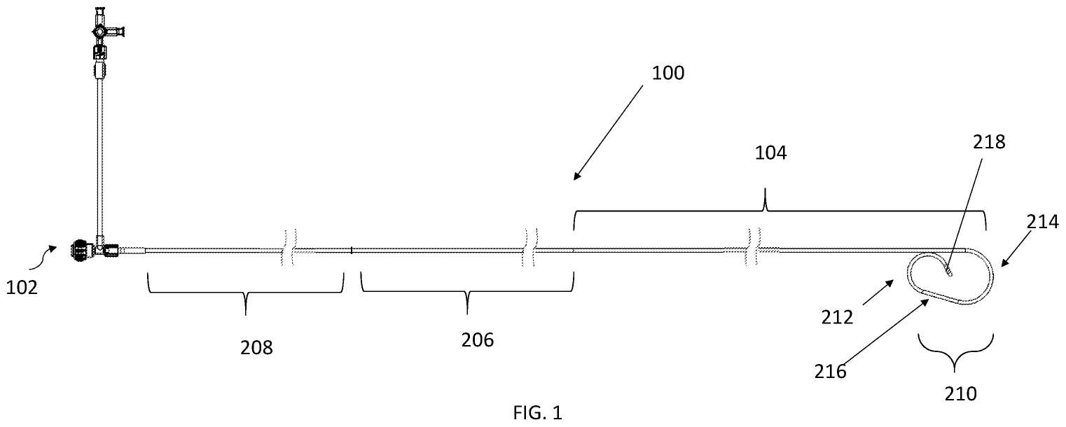

FIGS. 1-3L illustrate components that collectively comprise an embodiment of a conveyor system suitable for use in moving a TAVR delivery system intravascularly from a percutaneous entry position to its ultimate position within the heart. Further details concerning each of the illustrated components may be found in the various co-pending patent applications referenced in this section, each of which is incorporated herein by reference.

Right-to-Left Conduit

Referring to FIG. 1 the system includes a Right-to-Left conduit 100 (“RLC”), an elongate tubular catheter having a length sufficient to permit it to extend from the RFV of a human adult to the right atrium, across the interatrial septum (via a trans-septal puncture) to the left atrium, through the mitral valve, left ventricle, aortic valve to the aortic arch, and then to the descending aorta. In a preferred embodiment, this length exceeds 150 cm, and it may be 160 cm or longer.

A lubricious lumen extends through the RLC 100 from a proximal port 102 to an opening at the distal end 104. A flush port is also fluidly connected with the lumen of the RLC as shown. The RLC 100 is used to aid in the passage of other system components from the venous vasculature through the heart (including through a transseptal puncture) to the arterial vasculature, so that they can be used to deliver the TAVR delivery system. Its distal portion 104 is shape set into a curved configuration which, as discussed in application Ser. No. 13/578,374, entitled Conduit for Transseptal Passage of Devices to the Aorta, filed Sep. 22, 2019 (incorporated herein by reference), helps the distal end of the RCL pass into the mitral valve after it has crossed the interatrial septum from the right to the left side of the heart, and aids in orienting the distal opening of the RLC towards the aortic valve when the distal part of the RLC is in the left ventricle, allowing it to move safely into the aorta as will be better understood from the discussion of the method steps relating to FIG. 7.

Proximal to the distal portion 104 are an intermediate portion 206, and a proximal portion 208. The proximal and intermediate portions, 208, 206 and much of the distal portion 104, are of generally straight tubular construction. These parts of the shaft may be collectively referred to as the main body of the shaft. The distal portion 104 includes a distal loop 210 that has been shape set. The shape of the loop helps the distal end of the RCL pass into the mitral valve after it has crossed the intra-atrial septum from the right to the left side of the heart, further aids in orienting the distal opening of the RLC towards the aortic valve (as will be discussed in connection with the method below) when the distal part of the RLC is in the left ventricle.

More particularly, the distal loop 210 includes a distal (where for the purposes of this description of the curves of the RLC the term “distal” and “proximal” are used in regard to the entire length of the catheter) curve 212, a more proximal curve 214, a generally straight segment 216 extending between the curves, and a distal tip 218. The generally straight segment 216 may be straight or it may be curved with a very large radius of curvature to produce a significantly more gradual curve than the proximal and distal curves. While the term “straight” is used to refer to the shape of portions of the RLC in some embodiments, it should be pointed out that the catheter's inherent flexibility may cause it to bend under forces of gravity when held upright, or to curve when tracked over a curved cable or wire, or advanced into contact with another structure. The term “straight” thus should not be used to interpret this application or the corresponding claims as requiring the portion described as being “straight” to hold a straight shape when subjected to gravity or forces from another structure.

The curves 212, 214 are arranged to cause the distal loop 210 to curve back on itself, so that the distal curve 212 is formed by a part of the RLC shaft that is closer along the length of the shaft to the distal tip 218 than is the proximal 1 curve 214. The radius of the distal curve is smaller than that of the proximal curve.

The materials for the RLC are selected to give the conduit sufficient column strength to be pushed through the vasculature, torqued to orient its tip towards the aortic valve, and tracked over a wire, and it should have properties that prevent the distal loop 210 from permanently deforming as it is tracked over a wire. These properties are achieved using a sequence of regional durometers along the length of the RLC as described in U.S. Ser. No. 13/578,374. Although the distal loop 210 is moved out of its pre-shaped loop configuration to track over the wire, it is important that the shape-setting of the curves be retained. Otherwise the performance benefits of the distal loop's shape which, as evident from the Method description below are to aid proper movement into and through the mitral valve, to orient the tip of the RLC towards the aortic valve (so the wire can enter the aortic valve), and to track over the wire all the way to the descending aorta will not be realized.

It should be pointed out that while a number of preferred features for the RLC have been described above, alternative embodiments of the RLC might use any sub-combination of the above-described features alone or with other features not described here.

It will be further understood from the description of FIGS. 5 and 6 that in preferred methods of using the system, the RLC is advanced from the left ventricle across the aortic valve and into the aorta, establishing a conduit between the venous and arterial vasculature. This conduit is used as a guide for a cable that is to be positioned between the venous and arterial vasculature as discussed below.

The RLC may be steerable using pullwires or alternative means. In one embodiment, the distal curve 212 is steerable using one or more pullwires to facilitate its movement from the left atrium through the mitral valve to the left ventricle, and to then orient its distal tip towards the aortic valve. The proximal curve 214 may also be steerable, or the shape of its curve can be altered by manipulating the guidewire within it (i.e. withdrawing the guidewire from the curve 214 of the RLC to cause the curve to assume its native curved shape, advancing the guidewire into the curved section of the RLC to alter or straighten the curve. The various angles so created can be expanded or compressed to various degrees to accommodate cardiac chambers of different sizes by use of guidewires of varying stiffness within the RLC. Stiffer guidewires resulting in more expanded angles and more flexible guidewires resulting in more compressed angles. Even in these cases the regional durometers created by the materials described above in the different regions of the RLC are maintained.

Conveyor Cable

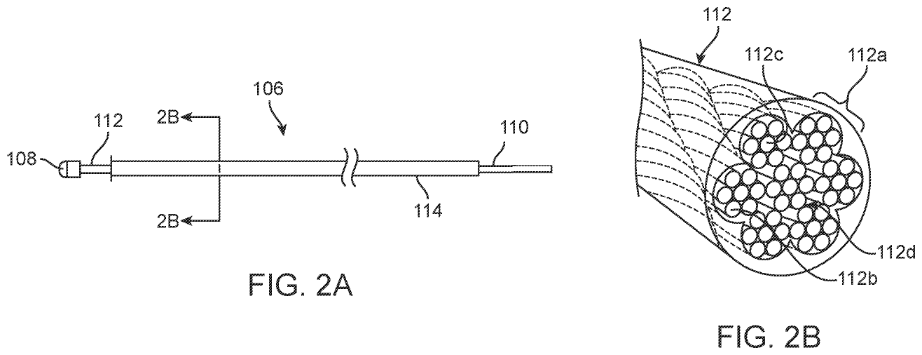

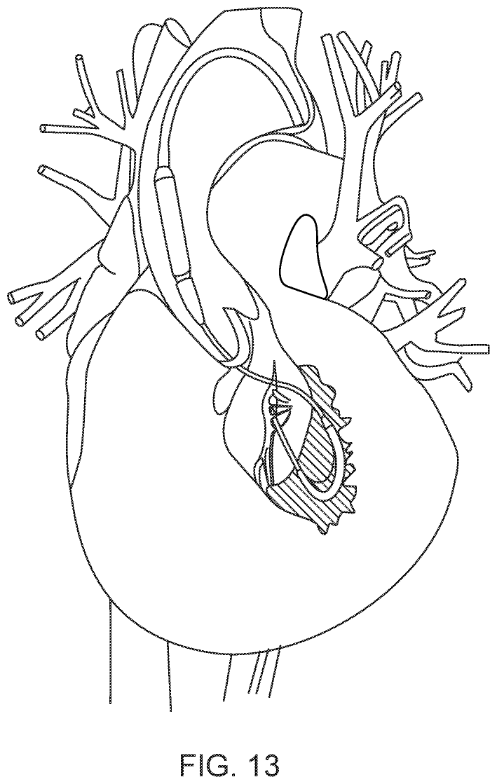

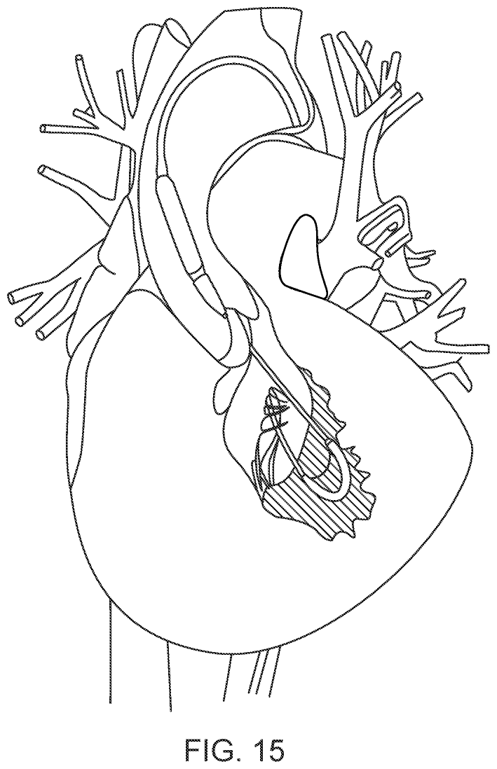

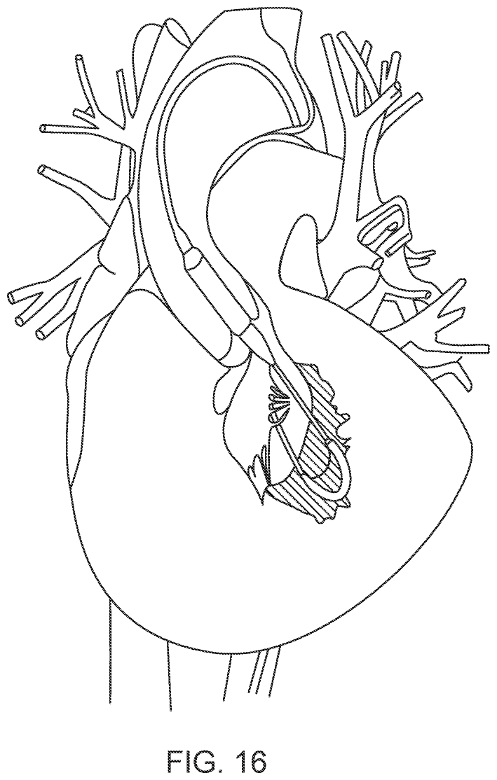

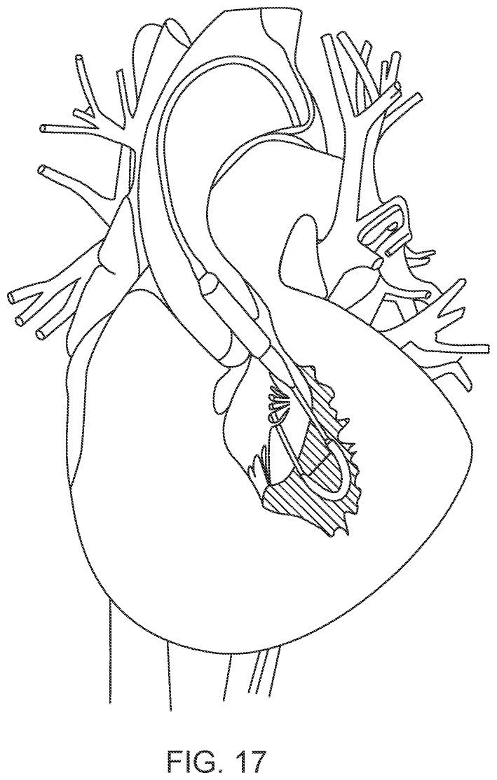

The system further includes a conveyor cable 106, shown in FIG. 2. The conveyor cable 106 comprises an elongate cable 112 having a first end with a first engageable feature 108 and a second end with a relatively stiff mandrel 110. The cable 112 is preferably made of a wire rope as shown in FIG. 2B but it can be any type of flexible tendon suitable for this purpose. The wire rope includes a plurality of strands 112a, each of which is formed of a plurality of individual wires 112b helically wound around a core 112c (which itself may be a wire). One wire of a strand 112a may comprise a core about which the other wires are wound as shown. Additionally, the wire rope itself has a core 112d about which the strands 112a are helically wound. In this particular example, the core 112d has the same or similar construction as a strand 112c. In alternative embodiments, the cable may instead be manufactured of stainless steel braid or another suitable material or construction having the column strength to be advanced through the RLC when pushed, and the tensile strength to be able to pull the distal end of the TAVR delivery system as traction is applied to the cable from articulation of the LVR as discussed in connection with FIGS. 13-17.

The cable is of sufficient length to extend from the right femoral vein (RFV) or other venous access point, through the heart via transseptal puncture, through the mitral and aortic valves, and through the aorta to the right or left femoral artery (RFA, LFA) of an adult human.

During the course of the method described below, one end of the conveyor cable 106 is captured and/or securely engaged by a snare, grasper or other engagement device. For this reason, feature 108 is designed so that it can be engaged using the engagement device provided for that purpose

In the embodiment shown in FIG. 2A, the engageable feature 108 is a tip element integrally positioned on a distal end of cable 112. Tip elements may take a variety of different forms. The one shown in FIG. 2A has a distal face with convex curvature and a cylindrical proximal part with a generally flat proximal face, a shape that facilitates engagement using a snare or grasper jaws. A lubricious polymer coating 114 (e.g. PEBA) covers the cable 112, leaving a gap between the end of the coating and tip element 108. This allows room between the tip element and coating 114 for an engagement device such as a snare or grasper jaw to seat when engaging the tip element.

Left Ventricle Redirector

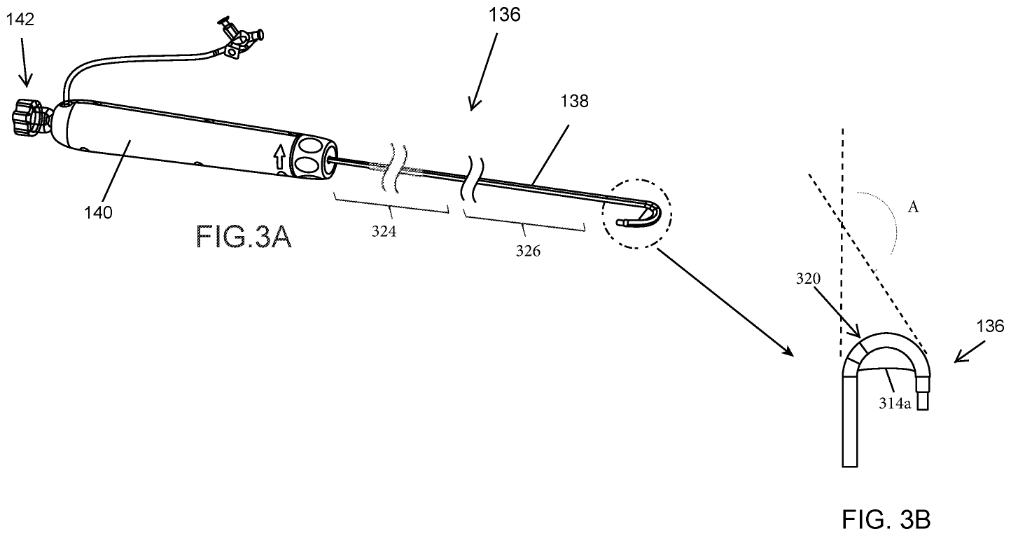

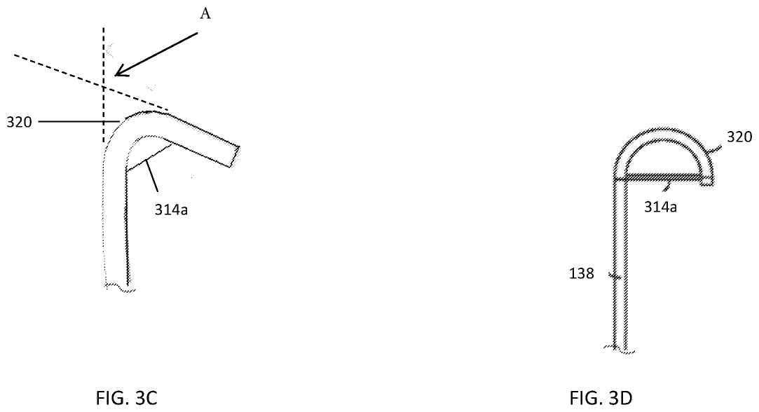

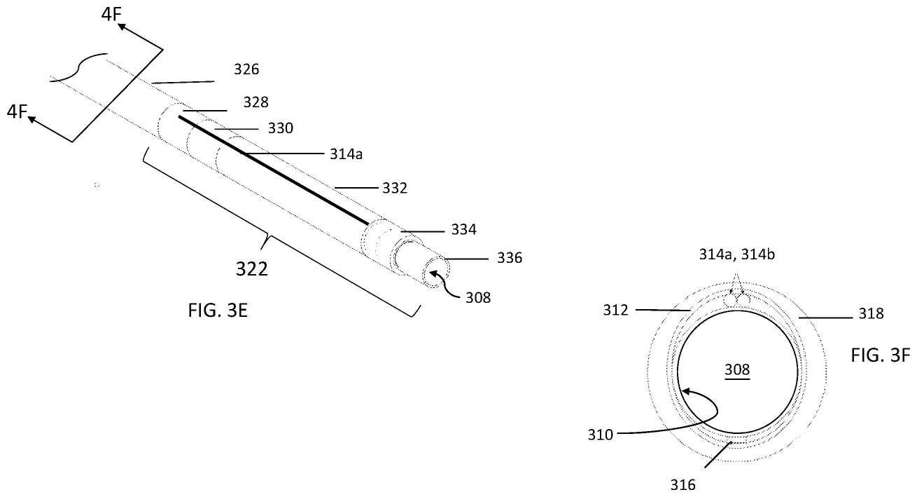

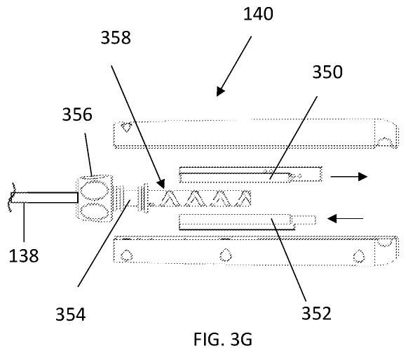

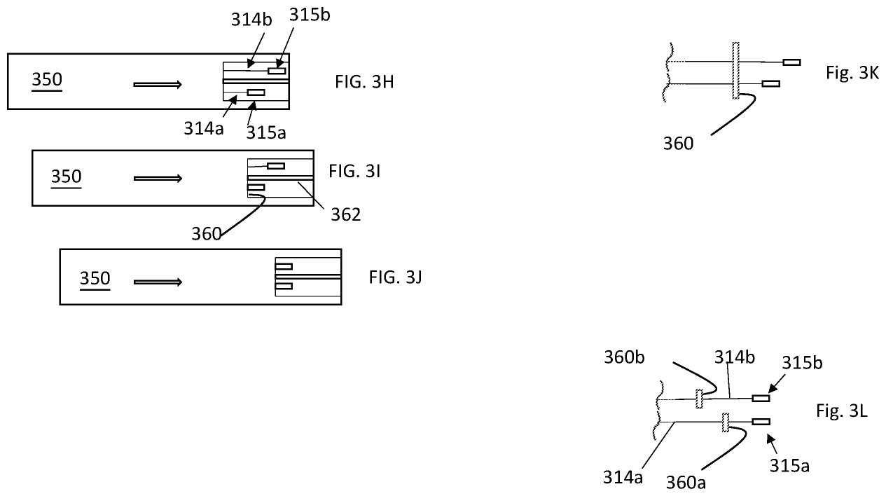

The system further includes a sheath that is positionable within the left ventricle and used to “redirect” the cable and TAVR delivery system. It is thus referred to as the “left ventricular redirector” or “LVR” 136. It is shown in FIGS. 3A and 3B and described in greater detail in application Ser. No. 16/578,379, entitled Device and Instrument for Facilitating Transseptal Delivery of Cardiac Therapeutic Devices, filed Sep. 22, 2019 (SYNC-5300), which is incorporated herein by reference. The LVR includes an elongate catheter shaft 138 having a proximal handle 140 with a proximal access port 142 and a flush port. The shaft includes a lumen 308 accessible via the access port 142. This lumen extends to the distal tip of the shaft.

FIG. 3F is a cross-section view showing the shaft construction. As shown, a lubricious layer such as an extruded PTFE liner 310 lines the wall of the lumen 308, and a braid 312 covers the liner 310. Incorporated between the liner 310 and braid 312 are a pair of pullwires 314a, 314 and a return wire 316. The pull wires 314a, 314b are directly adjacent to one another. Their side-by-side positioning causes bending of the LVR along a bending plane P1 when tension on the wires is increased.

The return wire is positioned 180° from the pull wires as shown. It may have a rectangular diameter with the long edges oriented to cause the shaft to preferentially bend along bending plane P1. One of the pullwires 314a exits and then re-enters the shaft towards the shaft's distal end. This will be explained in the description of FIGS. 3B and 3C.

An outer jacket 318 of polymeric material (e.g. polyether block amide, “PEBA,” such as that sold under the brand name Pebax) 314 covers the braid 312. During manufacture of the shaft, the polymeric material is positioned over the braid and subjected to a reflow process to flow the polymeric material over the braid. The material properties of the polymeric material vary along the length of the shaft. This is discussed below.

The distal end of the shaft is moveable between the generally straight position shown in FIG. 3C, and an articulated position in which the distal end is formed into a curve, as shown in FIG. 3B. The parts of the shaft that are proximal to the curve 320 may be collectively referred to as the main body of the shaft. The maximum articulation angle A is in the range of 100-140 degrees, with a more preferred range of 110-135 degrees, but may be different depending on the application for which the LVR will be used. In some embodiments, however, articulation of 180 degrees or higher may be achieved. The handle 140 (FIG. 1A) includes actuators to actuate the pull wires 314a, 314b to bend the shaft and to actuate the return wire 316 to return the distal end of the shaft to the generally straight configuration.

One of the pullwires 314a exits the sidewall of the shaft near the shaft's distal end, runs along the exterior of the shaft in a distal direction, and re-enters the shaft at the distal end of the shaft, while the other pull wire 314b does not exit the shaft at the distal end. The dual pull wire configuration advantageously allows articulation to the desired curvature and locking of the articulation in that curvature despite high loads experienced at the tip of the LVR during use.

The pull wire 314b that remains inside the shaft (“internal pull wire”) helps maintain the patency of the shaft's lumen during articulation, preventing the shaft from buckling or kinking despite the large degree of articulation as would likely happen if the construction used only the external pull wire.

The pullwire 314a that exits the shaft (the “external pull wire”) enables large degree articulation with forces sufficient to apply enough traction to the cable to steer the TAVR delivery system as discussed in connection with FIGS. 13-17.

Note that the terms “pullwire” and “wire” are not intended to mean that the pullwires must be formed of wire, as these terms are used more broadly in this application to represent any sort of tendon, cable, or other elongate element the tension on which may be adjusted to change the shape of the LVR. Also, while the term “straight” is used to refer to the shape of the LVR distal portion in its non-articulated position, it should be pointed out that the catheter's inherent flexibility in the non-articulated position may cause it to bend under forces of gravity when held upright, or to curve when tracked over a curved cable or wire, or advanced into contact with another structure. The term “straight” thus should not be used to interpret this application or the corresponding claims as requiring that portion of the LVR shaft to hold a straight shape.

The pullwire and return wire configuration shown in FIG. 3F also provides for steering in two directions, with movement occurring along one plane P1 between straight and curved positions. Other embodiments can be configured with additional directions of movement if desired.

The shape of the curve formed on actuation of the pullwires may differ for different embodiments. In the example shown in FIGS. 3B and 3D, the shaft curves about a relatively large radius as shown to produce a fairly shallow curve. In these embodiments, the straight portion of the distal end that extends beyond the curve 320 has a longitudinal axis that is parallel to the longitudinal axis of the main body of the shaft. In another embodiment shown in FIG. 3C, the shaft curves about a relatively small radius

The distance between the distal location at which the pullwire 314a re-enters the shaft and the distalmost end of the shaft tip may also vary between embodiments. In the FIGS. 3B and 3C embodiments, that entry site may be spaced 10 or more mm from the distalmost end of the shaft tip, whereas in the FIG. 3D embodiment, the entry site may be spaced 5 mm or less from it.

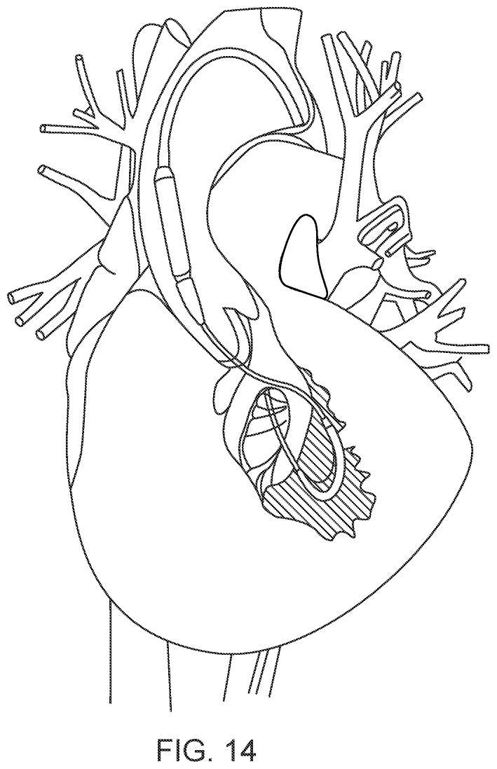

Material properties of the LVR components will next be described, although materials having different properties may be used without departing from the scope of the invention. The materials for the shaft are selected to give the LVR enough column strength to be pushed through the vasculature, torqued, and tracked over a cable or wire through the aortic arch, and articulated at the distal tip section 322 without kinking, and to allow the outer circumference of the curve formed when it is articulated to be pressed into the left ventricle away from the mitral and aortic valves as will be described in connection with FIG. 14. A wire braid 312 extends through the shaft 338 to enhance the torque ability of the LVR. A lubricious liner 310 made using PTFE, UHMWPE or like material also extends through the shaft, allowing smooth relative movement between the LVR and the wires or cables that pass through it during use. The braid and liner terminate in the distal tip section 322 as will be described with respect to FIG. 3E.

Referring to FIG. 3A, the shaft 338 is of sufficient length to extend from the left femoral vein or a radial vein, across the inter-atrial septum to the left atrium, through the mitral valve into the left ventricle (“LV”), so that the distal tip section 322 can be moved into its curved position, with the curve oriented towards, and optionally seated at, the LV apex. To meet this requirement, the length of shaft extending from the handle may have a length in the range of 900-1200 mm, and more preferably in the range of 1000-1100 mm. The materials used for the outer jacket 318 of the shaft vary along the shaft's length. The shaft includes a rigid section, formed of L25 nylon or similar material, that is disposed within the handle and that continues through the most proximal section 324 of the shaft lying outside the handle. The next most distal part 326 of the jacket is formed of fairly rigid polymeric of at least 72D shore hardness, to give the LVR the column strength needed for advancement through the vasculature. When the LVR is positioned with the curve in the left ventricle, this section externalizes through the introducer in the venous access point in the femoral vein. Within the handle, the jacket 318 may include both 72D PEBA and L25 Nylon, to further enhance column strength.

Referring now to FIG. 3E which shows the distal tip section in its straight position. The distal tip section 322 of the preferred embodiment includes five segments. In each of these segments the outer jacket 318 has different rigidity compared with the adjacent segments. The most proximal of these segments 328 is adjacent to section 326 described in the previous paragraph. It is a short section with a jacket of a fairly rigid material (e.g. 62D PEBA). It is through the wall of this segment that the pullwire 314a extends from inside the shaft to outside the shaft. The rigid material helps prevent tearing around the opening through which the pullwire passes 314a even when the pullwire is highly tensioned.

In the distally adjacent segment 130, a slightly less rigid material is used (e.g. 40D PEBA). This is done to provide a gradual transition between the rigid segment 328 and the next adjacent segment 332 which is highly flexible. The transition segment helps to avoid buckling.

Segment 332 is the longest segment within the distal tip section 322 and it is designed to facilitate bending of the shaft into the curve during articulation using the pullwires. It has a jacket made from a very flexible material (e.g. 80A Pelathane). The braid 312 (not shown in FIG. 3E) terminates at the distal end of this segment 332. The external pull wire 314a extends along the exterior surface of section 322 as shown in FIG. 3E.

Distally adjacent to flexible segment 332 is the segment 334 in which the pullwire 314a re-enters the shaft, and it is also the segment in which the pullwires 314a, 314b and return wire 316 are anchored to a pull ring (visible in dashed lines in FIG. 2). It is formed of a rigid material (e.g. 72D PEBA). The lubricious liner 310 may terminate at the distal end of segment 334.

The distal most segment 336 provides an atraumatic tip for the shaft. It is formed of highly flexible polymeric material, such as 35D PEBA having a sufficiently thick wall thickness and luminal diameter to be able to articulate against the cable without tearing.

In one embodiment, the polymeric material of the distal segment 334 is doped with BaSO4 to allow the tip of the LVR to be seen on the fluoroscopic image. Alternatively, a marker band made from radiopaque material may be positioned near the tip.

The flexural properties, and thus the stiffness, of the LVR are sensitive to the durometer of the extrusions forming the shaft, the reinforcement configurations used in the shaft (e.g. the braid and optional reinforcing wire, if used) and the geometry of the shaft. In a preferred embodiment made with the materials described above, an inner diameter of approximately 6 Fr and an outer diameter of approximately 9 Fr, the region rigidity of the shaft increases by a factor of approximately two as it transitions from region 326 proximally to the region 324 that extends to the handle, giving the LVR column strength that will allow it to be pushed through the vasculature as described in connection with its method of use.

A discussion of the actuation mechanism for the pull wires 314a, 314b and return wire 316 will next be described. In general, the handle 140 is configured to move the pull wires 314a, 314b in a first direction (preferably proximally) while simultaneously moving the return wire 316 in a second, opposite direction (preferably distally), in order to articulate the LVR to the curved position. Reversing the respective directions of motion of the pull wires 314a, 314b and return wire 316 moves the LVR back to the generally straight position.

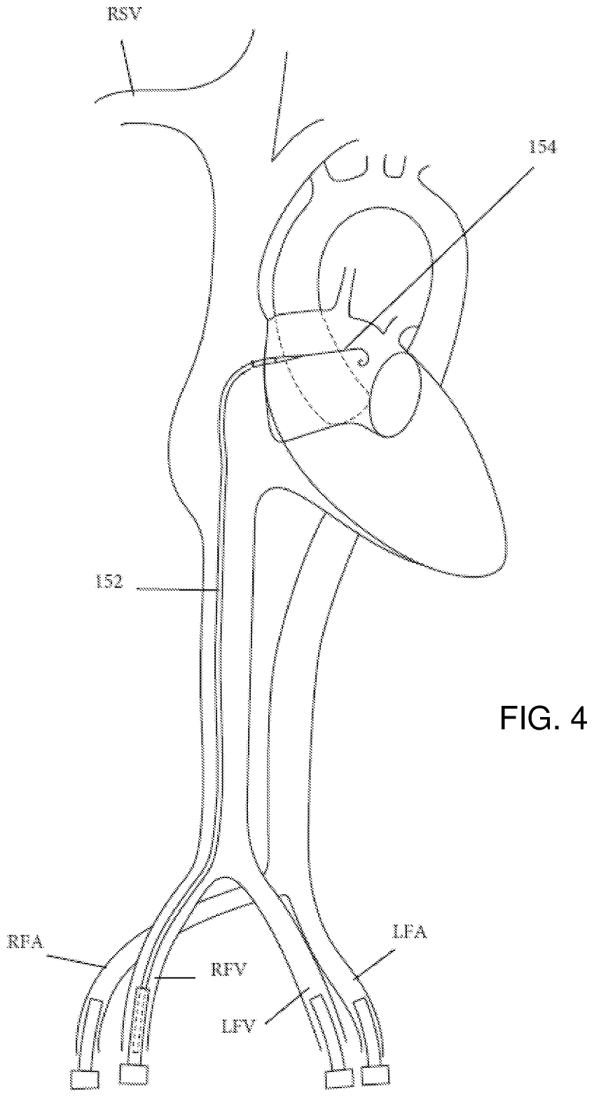

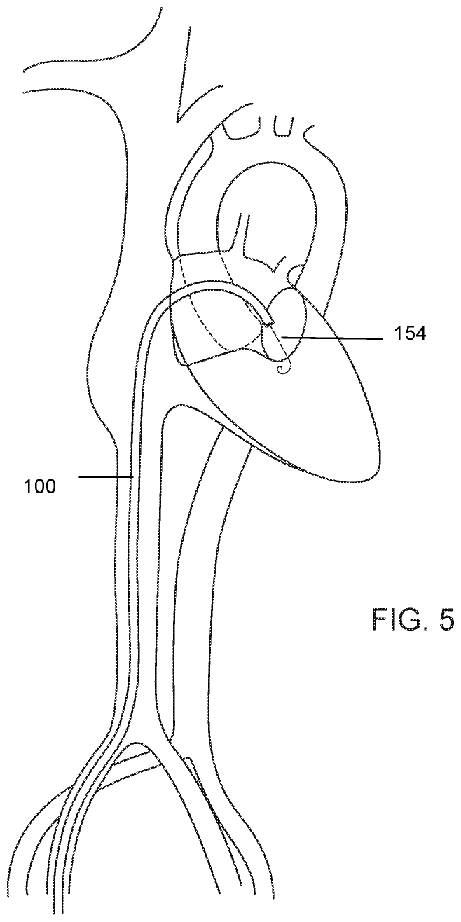

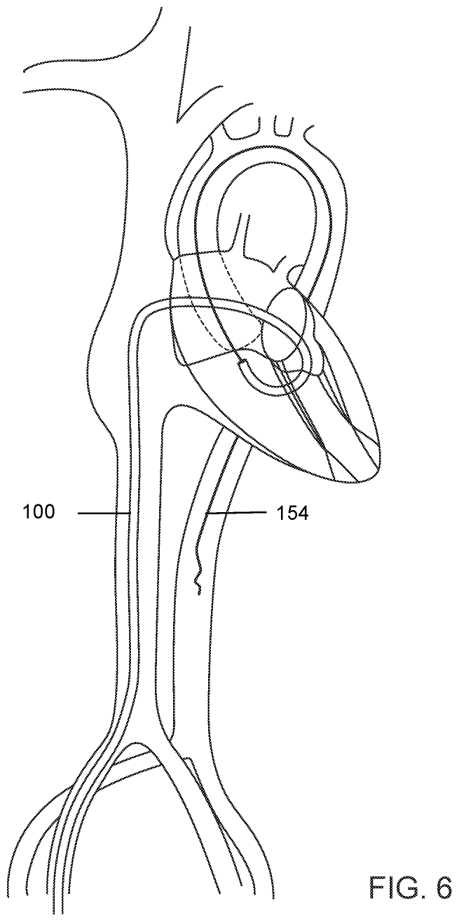

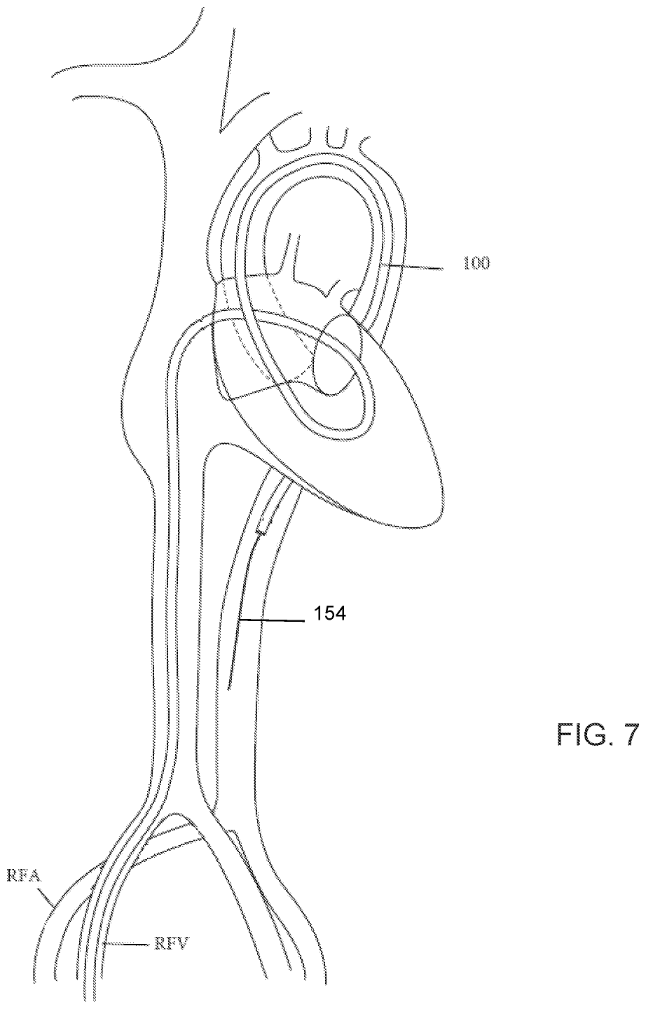

Referring to FIG. 4G, within the handle 140 are a first sliding member 350 and a second sliding member 352, each of which is moveable longitudinally within the handle. For convenience the term “slider” will be used as shorthand for “sliding member.”

The handle 140 includes a mechanism for simultaneously moving the sliders 350, 352 in opposite directions. Various mechanisms can be used for this purpose. One exemplary mechanism, shown in FIG. 4G, includes a barrel 354 that axially rotates relative to the handle 140 when the user rotates actuation knob 356. Helical features 358 on the barrel 354 are operatively associated with helical features (not visible in the drawings) in each slider 350, 352, with the helical features 358 that interact with slider 350 being of opposite hand to those that interact with slider 352. Thus, rotation of the knob 356 in a first direction causes slider 350 to move proximally while slider 352 moves distally (see arrows in FIG. 4G), and rotation of the knob 356 in the opposite direction causes slider 350 to move distally while slider 352 moves proximally. The pull wires 314a, 314b (not shown in FIG. 4G) extend into the handle 140 from the LVR shaft 138 and are actuatable by first slider 350, while the return wire 316, which also extends into the handle from the LVR shaft, is actuatable by the second slider 352. With this configuration, motion of the sliders 350, 352 causes both pull wires 314a, 314b to be pulled as the return wire 316 is simultaneously pushed, and vice versa. Note that while the knob 356 and barrel 358 assembly is a convenient way to actuate this motion using a single input from the user, alternative mechanisms can be used without departing from the scope of the claims.

The two pull wires 314a, 314b must travel different distances during articulation, due to the fact that the internal pull wire 314b traverses the curve resulting from the articulation from its position within the shaft, while the external pull wire 314a traverses a shorter path between the point at which it exits the shaft and the point at which it re-enters the shaft. The wires must therefore be actuated at different positions within the handle so as to ensure that the external pull wire 314a maintains equal or greater tension than the internal pull wire 314b. This avoids wire slack and ensures that the locking mechanism does not relax during application of forces F at the LVR's tip.

FIGS. 3H-3J are a sequence of drawings illustrate a configuration for the slider 350 that is designed to actuate the pull wires 314a, 314b from different positions. Each pull wire 314a, 314b is shown with an actuation feature 315a, 315b at its proximal end. The actuation feature can be any feature that will be engaged by a corresponding feature of the slider 350 as the slider moves into contact with the actuation feature. In this embodiment the actuations feature 315a, 315b are elements, on the proximal ends of the pull wires, that have diameters wider than the diameter of the pull wire itself. In this particular example, the actuation feature for each wire is its corresponding crimp.

Distal to each actuation feature 315a, 315b is a corresponding feature of the slider that will engage that actuation feature as the slider 350 moves in the proximal direction (indicated by the arrow in FIG. 3H). In the embodiment depicted in the drawings, the pull wires 314a, 314b have their proximal ends positioned proximal to a proximally facing face 360. The pull wires 314a, 314b may extend through openings in the member on which the face is position as illustrated in FIG. 3K. Each opening is smaller in diameter than the diameter of the actuation feature 315a, 315b. The face 360 serves to engage the actuation feature 315a, 315b of each pull wire 314a, 314b as the slider 350 moves proximally. Note that there may be two separate members having faces 360a, 360b (see FIG. 4L), one for each of the pull wires 314a, 314b, or the face 360 may be divided by a longitudinal barrier 362 that maintains separation between the proximal ends of each pull wire as shown in FIGS. 3H-3J.

FIG. 3H depicts the slider 350 and pull wires 314a, 314b when the LVR is in the generally straight (non-articulated) position. As shown, the length of the external pull wire 314a is shorter than that of the internal pull wire 314b, so that when the slider 3150 moves proximally, the external pull wire 314a is actuated by engagement feature/surface 360 first (FIG. 3I), and it is then pulled a predetermined distance before the internal pull wire 314b is actuated by engagement feature 360 (see FIG. 3J, which shows the pull wires after actuation of the internal pull wire 314b has begun). This ensures that the loading of the external pull wire is higher than, or at least equal to, the external pull wire, as discussed above. From the position shown in FIG. 3J, proximal movement of the slider continues until the pull wires 314a, 314b bring the shaft to its fully articulated position.

In an alternative arrangement shown in FIG. 4L, the slider may have separate features 360a, 360b positioned to actuate the pull wires 314a, 314b at different points along the slider's travel. FIG. 4L depicts these features of the slider and the pull wires when the LVR is in the generally straight (non-articulated) position. The features 360a,360b are positioned on slider so that as slider moves proximally, feature 360a engages external pull wire 314a (e.g. at crimp 315b or other actuation feature as discussed above) and pulls it a predetermined distance before the feature 360b similarly actuates internal pull wire 314b.

In each of the above actuation embodiments, the distance by which external pull wire 314a will travel before internal pull wire 314b is engaged is selected to be the approximate difference between L1 and L2. In this calculation, L1 is the length of external pull wire 314a between its exit and entry points into and out of the shaft when the LVR is in the fully articulated position. L2 is the length traversed by the internal pull wire 314b along the internal circumference of the curve, measured between the points on the internal pull wire's path that are circumferentially adjacent to the points at which the adjacent external pull wire exits and then re-enters the shaft.

### Method of Use

A method of using the system to deliver a TAVR delivery system carrying a TAVR device to its operative location within the heart will next be described with reference to FIGS. 4-18.

As an initial step, the practitioner obtains percutaneous access to the vessels that will be used in the procedure using percutaneous access sheaths. This will include the right femoral artery (RFA) and or the left femoral artery (LFA), the right femoral vein (RFV-11 F sheath) and/or the left femoral vein (LFV-11 F sheath), or any of the other vessels discussed above. FIG. 4 shows percutaneous access sheaths positioned in each of the RFA, LFA, RFV and LFV vessels. Access sheath sizes are selected in sizes suitable for the devices that are to be passed through them.

A Brockenbrough transseptal catheter (BTC) 152 is introduced through the RFV and, using the well-known technique of transseptal catheterization, is passed from the right atrium (RA) into the left atrium (LA). The right and left atrium are not labeled in FIG. 4. A wire 154, which may be an 0.035″ wire such as the Abbott Versacore wire, is passed through the BTC and into the left atrium (LA).

The BTC 152 is withdrawn at the RFV and exchanged for an RLC 100 which is advanced over the wire. See FIG. 5. The RLC which preferably has been filled with an 80/20 saline-contrast solution for additional visibility under fluoroscopy. After it has crossed the interatrial septum into the LA, the RLC 100 is advanced toward the lateral edge of the LA, and the wire 154 is pulled back into the RLC, proximal to the loop 210 (see FIG. 1) of the RLC. The RLC is rotated counterclockwise about the axis of the main body of its shaft as the RLC is slowly withdrawn. This motion causes the tip of the RLC to drop in an inferior direction into and through the mitral valve MV towards the left ventricle. Once the tip is through the MV, the RLC continues to be advanced, its shape causing the distal end of the tip to move in a right-ward (the patient's right) and anterior direction. Some rotation of the RLC may be needed in this step to get the tip towards the AV. This direction of motion helps orient the RLC's tip towards the aortic valve, since the aortic valve is anterior and to the right of the mitral valve.

The RLC's curvature as well as active steering of the distal end can most efficiently directs its tip towards the aortic valve. Where the RLC is provided with a steerable distal curve 212 (FIG. 1), the distal end is actively steered by increasing tension on its pullwire(s) to orient the distal tip towards the aortic valve. FIG. 6 shows the distal tip of the RLC pointed towards the aortic valve. As shown, section 104 of the RLC extends within the inferior vena cava, extends through the interatrial septum (not shown), drops into the mitral valve and forward into the left ventricle. Note with regard to this drawing that the distal curve 212 is positioned anterior (out of the plane of the drawing towards the viewer) with respect to the proximal curve 214.

It should also be mentioned that movement of the RLC through the heart as described above is optimally performed using a variable stiffness guidewire, allowing the variations in curvature and stiffness along the length of the RLC to work together with the different degrees of regional stiffness of the guidewire. A suitable variable stiffness guidewire is one having at least three segments of different flexibility. The first, and most distal of those segments has the greatest flexibility. A second segment is proximal to the distal segment and has less flexibility than the first segment, and a third segment is proximal to, and less flexible than, the second segment. In one specific example, the first and third segments are directly adjacent to the second segment.

Where a variable stiffness guidewire is used, during the step of crossing the septum with the RCL, the stiffest segment of the guidewire is positioned through curves 212, 214 of the RLC, forming it into a gently curved configuration. In this more straightened configuration, advancement of the RLC, after it crosses the septum, causes its tip to cross the left atrium to a position beyond the mitral valve, and optionally in a left pulmonary vein. After the RLC reaches this position, the guidewire is withdrawn so the most flexible distal section, at least within the curve 212 of the RLC, causing the RLC to return to a more curved orientation due to the withdrawal of the stiff part of the guidewire from the loop 210 of the RLC. Counterclockwise torque is then applied as the RLC is withdrawn, causing the RLC tip to move anteriorly through the mitral valve. The tip will drop from the mitral valve into the left ventricle. The RLC is pushed with clockwise torque, or with alternating clockwise and counterclockwise torque, while the RLC is actively steered at the distal curve 212 using its pullwire mechanism to direct the RLC tip adjacent to the ventricular septum and pointing to the left ventricular outflow tract. Next, the guidewire is advanced through the aortic valve and around the aortic arch, allowing the RLC to be advanced on the stiffer segment of the guidewire.

With the RCL positioned in this way, the guide wire 154 is advanced through the aortic valve, around the aortic arch, and well down into the descending aorta, as shown in FIG. 7. The wire and RLC are advanced through the descending aorta and optionally into the RFA.

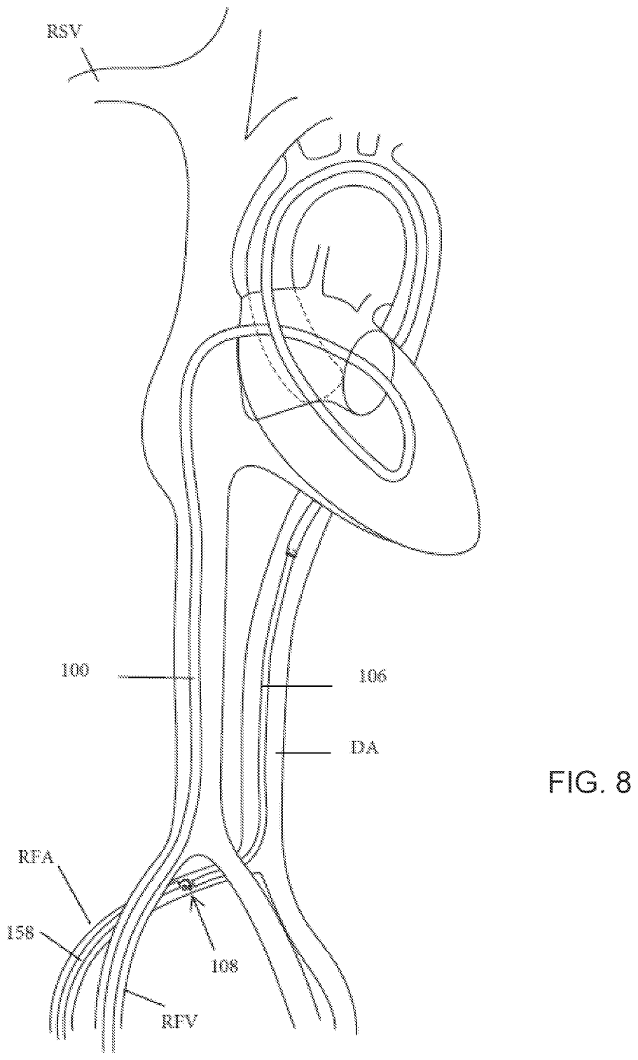

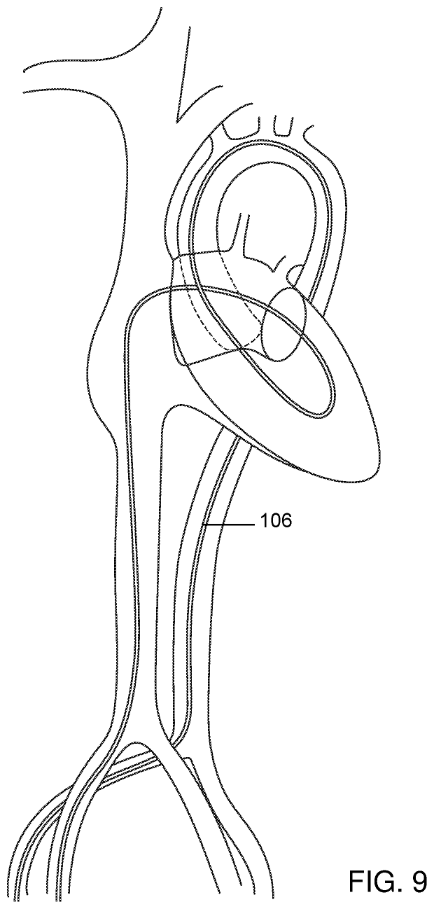

The wire 154 is withdrawn from RLC at the RFV and exchanged for the cable 106. The cable is inserted into to RLC at the RFV with the first engageable feature 108 first. When the cable 106 emerges from the RLC 100 in the descending aorta (DA) or RFA, a snare 158 advanced from the RFA and positioned on the cable. It is tightened around the cable 106, capturing it at the tip feature 108, and the snare is then withdrawn from the RFA to draw the end of the cable out of the body at the RFA. See FIG. 8.

The RLC is withdrawn, leaving the cable in place forming an AV loop as shown in FIG. 9.

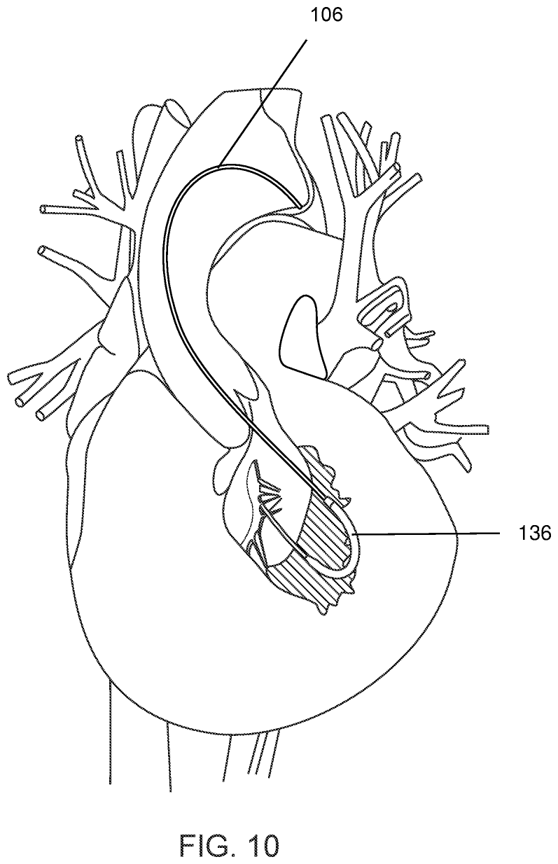

The portion of the cable 106 extending from the venous side (in this embodiment at the RFV), is backloaded through the LVR 136 on the operating table so that the proximal end of the cable extends from the proximal handle of the LVR 136. Next, while not illustrated in the drawings, the steerable RLC is introduced temporarily over the cable 106 into the RFA and guided through the aorta, across the aortic valve, through the LV and into the LA, where it is engaged with the tip of the LVR once the LVR has been advanced from the RFV sheath the interatrial septum. This engagement may be carried out by inserted the tip of one of the LVR and RLC into the lumen at the tip of the other. This provides a smooth passage of the LVR through the LA and across the mitral valve into the LV. See FIG. 10 showing the LVR in position in the LV after the RLC has been withdrawn from the heart and out of the RFA sheath.

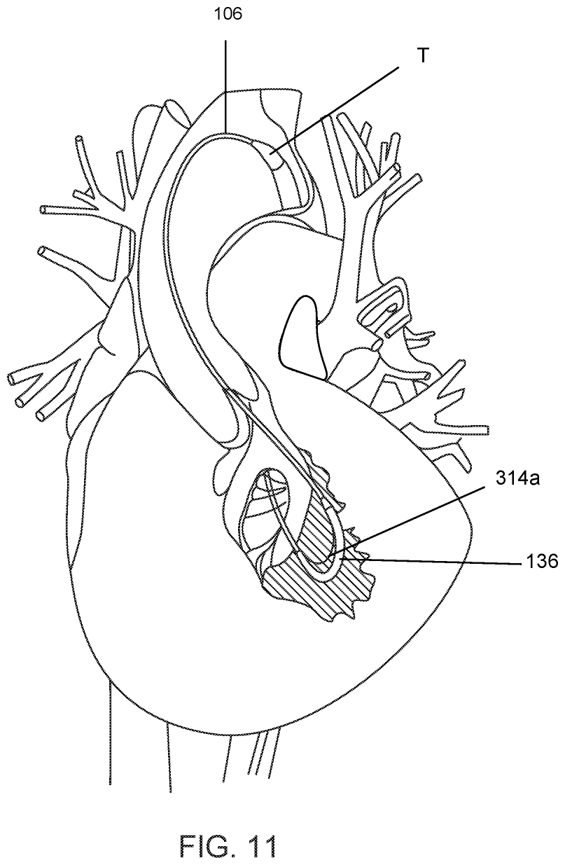

The portion of the cable 106 exteriorized at the RFA is backloaded through the TAVR delivery system on the operating table so that the tip 108 of the cable extends from the proximal end of the TAVR delivery system. The TAVR delivery system T is advanced over the cable 106 into the aorta. The LVR 136, still in the left ventricle, is deployed to its curved orientation. See FIG. 11. Forces imparted on the cable 106 by the deployed LVR aid in keeping the cable away from the surrounding conduction system, including the subvalvular conduction system.

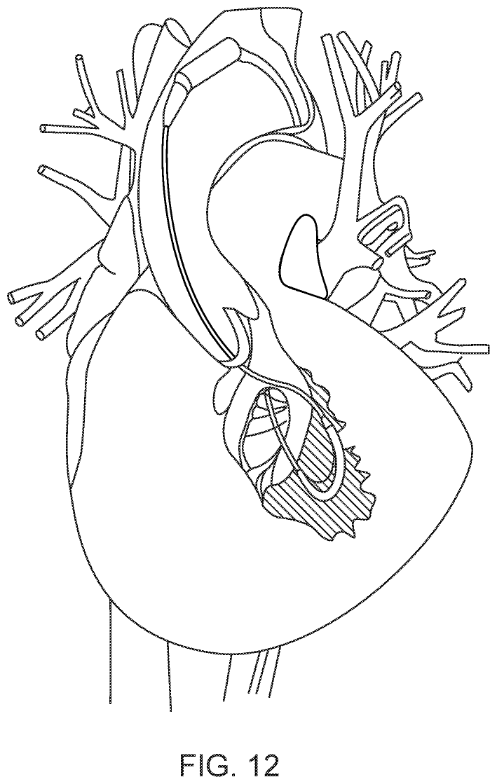

With the LVR in place in the LV, the TAVR delivery system is advanced over the cable to the aortic valve site. The steering mechanism of the LVR is used to precisely align the prosthesis for deployment, so it may be positioned clear of structures relating to the conduction system, such as the His bundle and the atrioventricular node. More specifically, once the TAVR delivery system is in the ascending aorta, the steering mechanism of the LVR is engaged to steer the distal tip of the LVR in order to apply traction to the cable, which imparts forces to the distal portion of the TAVR delivery system, steering it from its distal end as it moves into the center of the aortic valve. See FIGS. 12 through 17. Thus, by applying a steering force to the front of the TAVR delivery system the TAVR valve can be aligned coaxially in the center of the aortic valve orthogonal to the plane of the aortic valve annulus. This action avoids deployment of the valve at an angle to the annulus which often can result in a paravalvular leak. The LVR simultaneously pulls the distal tip away from the subvalvular conduction system to help avoid damage to the conduction system which can often result in complete heart block. The LVR also simultaneously helps to protect the left ventricular apex from the TAVR delivery system tip or wire perforations.

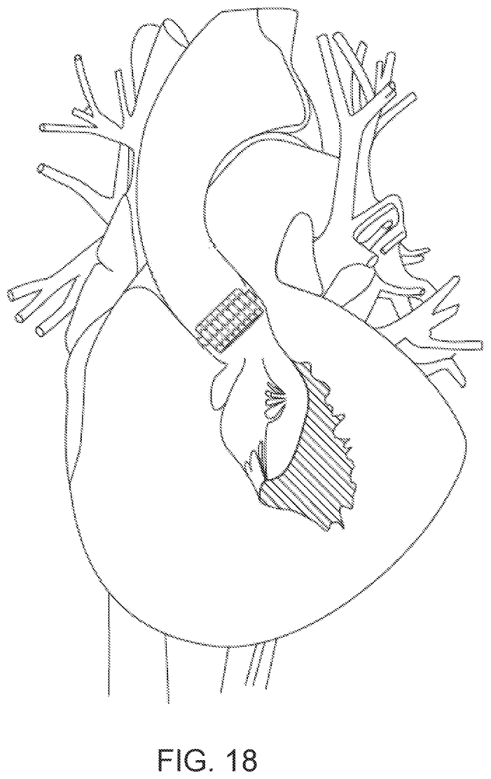

Once the operator is satisfied with the deployment of the prosthetic valve, the conveyor cable and valve delivery system are removed from the arterial side and the LVR is removed from the venous side, leaving the deployed valve in place. See FIG. 18.

Each of the patents and applications referred to herein, including for purposes of priority, are incorporated herein by reference.

What is claimed is:

1. A method for delivering a therapeutic device to a target aortic valve site, comprising:
(a) positioning a cable in a body of a patient to run between a femoral vein, through the heart via the right atrium, left atrium, mitral valve, aortic valve and into the aorta and arterial vasculature with the flexible cable having a first end extending outside the body from a venous vessel and a second end external to the patient at the femoral artery;
(b) advancing an aortic valve therapeutic device over the cable into a femoral artery to an aorta;
(c) advancing an actively steerable sheath over the cable into the venous vasculature and into the left ventricle of the heart by joining together at the level of the left atrium with a second steerable catheter introduced from the arterial side retrograde through the left ventricle to the left atrium
(d) actively articulating the sheath to form its distal end into a curved position within the left ventricle after the second steerable catheter has been withdrawn from the body;
(e) while maintaining the sheath in the curved position, pushing the aortic valve therapeutic device over the cable in a distal direction to advance the aortic valve therapeutic device from an ascending aorta to the target site.

2. The method of claim 1, further including steering the sheath during step (e) to apply traction to the portion of the cable at the distal end of the aortic valve therapeutic device, the traction steering the distal end of the aortic valve therapeutic device.

3. The method of claim 2, further including applying the traction to align and center the aortic valve therapeutic device at the target site.

4. The method of claim 1, wherein step (d) positions the portion of the cable extending through the aortic valve site in spaced relationship to structures of the conduction system of the heart.

5. The method of claim 4, wherein the structures are subvalvular structures.

6. The method of claim 1, wherein in step (a) the cable is positioned to extend from a femoral vein to a femoral artery.

7. The method of claim 1, wherein step (a) includes:
(i) advancing a right-to-left conveyor (RLC) into the vasculature from a femoral vein, and advancing the RLC to the right atrium and through a transseptal puncture into the left atrium, the RLC having a lumen with a distal opening;
(ii) advancing the RLC from the left atrium through the mitral valve into the left ventricle;
(iii) advancing the RLC while actively steering a distal curve of the RLC to cause the distal opening to orient towards the aortic valve;
(iv) advancing a wire through the RLC through the aortic valve to the descending aorta;
(v) advancing the RLC over the wire to the descending aorta;
(vi) withdrawing the wire from RLC at the femoral vein,
(vii) introducing a cable into the RLC at the femoral vein and advancing a first end of the cable to the descending aorta;
(viii) capturing the first end of the cable in the descending aorta or right femoral artery and withdrawing it from the right femoral artery.

8. The method of claim 7, wherein step (iii) includes rotating the RLC.

9. The method of claim 1, wherein the aortic valve therapeutic device is an aortic valve delivery system.
